(19) United States
(12) Patent Application Publication
(10) Pub. No.: US 2014/0220069 A1
(43) Pub. Date: Aug. 7, 2014

(54) ACTIVE INGREDIENT DERIVED FROM TORULASPORA DELBRUECKII AND COSMETIC USE FOR IMPROVING AND/OR REPAIRING THE BARRIER FUNCTION OF THE SKIN

(71) Applicant: Jean Paufique, Objat (FR)

(72) Inventor: Jean Paufique, Objat (FR)

(73) Assignee: SOCIETE INDUSTRIELLE LIMOUSINE D'APPLICATION BIOLOGIQUE, Objat (FR)

(21) Appl. No.: 14/342,968

(22) PCT Filed: Sep. 4, 2012
(86) PCT No.: PCT/FR2012/051977

§ 371 Date: Mar. 5, 2014

(30) Foreign Application Priority Data
Sep. 5, 2011 (FR) 11 57843

(51) Int. Cl. A61K 8/99; A61Q 19/00

(52) U.S. Cl. CPC A61K 8/99; A61Q 19/00
USPC 424/195.16; 530/371

(57) ABSTRACT

An active ingredient derived from Torulaspora delbrueckii and the use thereof for improving and/or restoring the barrier function of the skin. Also, cosmetic compositions containing this active ingredient and a cosmetic care process for improving and/or restoring the barrier function of the skin.

This invention relates to the cosmetic use of Torulaspora delbrueckii as a cosmetic active ingredient, in particular for improving and/or repairing the barrier function of the skin, as well as a cosmetic skin care process.

The invention also relates to an active ingredient that is derived from Torulaspora delbrueckii, to a production process, as well as to cosmetic compositions containing this active ingredient.

The barrier function of the skin is essential to maintaining hydration and physical properties of the cutaneous tissue. It results from epidermal differentiation, a sequential and oriented process during which the keratinocytes undergo numerous morphological and metabolic changes ending in the stratification of the epidermis and the formation of the horny layer.

The skin is continually subjected to attacks that lead to the destructuring of its barrier function.

The alteration of the cutaneous barrier has repercussions on the condition of the skin. It can be reflected in particular by a hydration problem: the skin loses its impermeability, it becomes dehydrated, and its negligible water loss increases.

The patent FR-0953114 describes an effective solution for improving and/or repairing the cutaneous barrier function by using—in a cosmetic composition—at least one agent that increases the expression of nicotinic acetylcholine receptors, nAChRs, of the skin cells.

The objective of this invention is to propose a specific active ingredient that exhibits such an effect.

To respond to this, the purpose of the invention is the use of an active ingredient that is derived from Torulaspora delbrueckii.

Torulaspora delbrueckii is a yeast that is isolated from marzipan. It is part of the branch of ascomycetes, the class of saccharomycetes, the order of saccharomycetales, the family of saccharomycetaceae, the genus Torulaspora, and the species delbrueckii. It is also known under the following synonyms: Saccharomyces delbrueckii, Saccharomyces fermentati, and Saccharomyces rosei.

This yeast is used in the food-processing industry for its fermentability.

However, surprisingly enough, Torulaspora delbrueckii has significant effects as regards the skin with very advantageous cosmetic properties.

The object of the invention is therefore an active ingredient derived from Torulaspora delbrueckii such as a cosmetic active ingredient in a composition that is intended to be applied on the skin for improving and/or repairing the cutaneous barrier function.

The object of the invention is also specifically a cosmetic active ingredient that comprises structural peptides obtained from Torulaspora delbrueckii as well as a process for the production of this active ingredient and cosmetic compositions incorporating at least one such active ingredient.

Preferably, the active ingredient according to the invention is obtained by hydrolysis of Torulaspora delbrueckii.

Finally, the invention also has as its object a cosmetic skin care process for improving and/or repairing the cutaneous barrier function.

This invention is now described in detail.

### Use

The invention relates to an active ingredient derived from Torulaspora delbrueckii for its application as active ingredient in a composition for topical application. In particular, the purpose of the invention is the cosmetic use of an active ingredient derived from Torulaspora delbrueckii as a cosmetic active ingredient in or for the preparation of a cosmetic composition.

In terms of the invention, active ingredient is defined as any molecule or molecule mixture that has cosmetic effectiveness on the skin and that is not a vehicle.

An active ingredient derived from Torulaspora delbrueckii or obtained from Torulaspora delbrueckii in terms of the invention is defined as any molecule or molecule mixture derived from the structure of the Torulaspora delbrueckii fermenting agent: this can involve native molecules of the yeast or molecules obtained by hydrolysis of the yeast. Preferably, it involves a Torulaspora delbrueckii hydrolyzate. The term active ingredient within the meaning of the invention excludes the molecules produced by fermentation of Torulaspora delbrueckii.

“Hydrolyzate” is defined as any extract that is obtained from Torulaspora delbrueckii, comprising at least one enzymatic or chemical, preferably enzymatic, hydrolysis stage.

The purpose of the invention is in particular the use of an active ingredient derived from Torulaspora delbrueckii as a restructuring cosmetic active ingredient.

According to the invention, an active ingredient derived from Torulaspora delbrueckii can actually be used as a cosmetic agent on the skin for improving and/or repairing the cutaneous barrier function. The presence of a sufficient quantity of peptides, in particular peptides with a molecular weight of less than 2,000 Da, in the active ingredient of Torulaspora delbrueckii makes it possible in particular to impart to it this cosmetic activity.

When it is applied on the skin, the active ingredient derived from Torulaspora delbrueckii acts in particular on the pathway of nicotinic acetylcholine receptors, nAChRs, of the skin.

The nAChRs are ion channels whose opening is regulated by acetylcholine. They are specialized in the transmembrane transport of cations and control numerous processes involved in the homeostasis of the epidermis. The stimulation of the nAChRs induces the influx of calcium and sodium and the efflux of potassium, which makes it possible to carefully regulate the intracellular ion flows and to synchronize the metabolic events taking place throughout the epidermal differentiation.

Monitoring the influx of calcium into the keratinocytes, the nACHRs greatly intervene in the regulation of the cohesion and the differentiation of keratinocytes, processes extensively involved in the formation of an optimum barrier function.

Advantageously, according to the invention, the use on the skin of an active ingredient derived from Torulaspora delbrueckii makes it possible to stimulate the nAChRs and thus to promote the epidermal homeostasis.

The cohesion of the epidermis rests on the presence of complexes incorporating adhesive junctions of the epidermis, which ensure a strong adhesion between the keratinocytes and which are necessary for the forming and maintaining of the barrier function. These junctions unite the actin cytoskeleton of two adjacent keratinocytes by means of a transmembrane glycoprotein located in the cytoplasmic membrane of the cells, the E-cadherin. In addition to their role as intercellular anchoring points, these junctions also operate as signaling platforms controlling the reworking of the cytoskeleton, the cellular migration, and the polarization of the epidermis.

Advantageously, according to the invention, the application of an active ingredient derived from Torulaspora delbrueckii on the skin makes it possible to stimulate the interkeratinocytic adhesion, in particular by stimulating the E-cadherin synthesis.

During their migration toward the cutaneous surface, the keratinocytes undergo biochemical and structural modifications for gradually transforming into keratinized cells. The nAChRs are involved in the terminal differentiation and the cornification of keratinocytes. They influence in particular the expression by the keratinocytes of cytokeratins 1 and 10, filaggrin, and proteins involved in the formation of the horny cover such as involucrin, loricrin, and transglutaminase-1.

According to the invention, the application of an active ingredient derived from Torulaspora delbrueckii on the skin makes it possible to stimulate the terminal differentiation of keratinocytes, in particular by stimulating the synthesis of loricrin.

The object of the invention is therefore an active ingredient derived from Torulaspora delbrueckii for its application as active ingredient in a composition for topical application, with the active ingredient and/or the composition being used to stimulate the nAChRs that are present on the membranes of the keratinocytes and thus to regulate the epidermal homeostasis. Specifically, the active ingredient according to the invention and/or the composition incorporating it can be used for:
Stimulating the interkeratinocytic adhesion, in particular by stimulating the synthesis of E-cadherin,
Stimulating the differentiation of keratinocytes, in particular by stimulating the synthesis of loricrin.

These actions have the effect of accelerating the construction of the epidermis, promoting its cohesion and its maturation, and thus improving the barrier function and the surface condition of the skin.

According to a particularly suitable embodiment, the purpose of the invention is the use of an active ingredient derived from Torulaspora delbrueckii containing peptides with a molecular weight of less than 2,000 Da, as described below.

### Active Ingredient

The purpose of the invention is therefore the use of an active ingredient derived from Torulaspora delbrueckii as an active ingredient in a composition for topical application. The invention also relates to a particular cosmetic active ingredient comprising peptides obtained from Torulaspora delbrueckii, preferably an active ingredient comprising peptides with a molecular weight of less than 2,000 Da obtained from Torulaspora delbrueckii.

Preferably, the active ingredient according to the invention comprising peptides obtained from Torulaspora delbrueckii is a hydrolyzate of Torulaspora delbrueckii, and even more preferably an enzymatic hydrolyzate of Torulaspora delbrueckii.

In a preferred way, the active ingredient according to the invention comprises a peptide content that is greater than or equal to 42% relative to the total content of dry material, preferably greater than 60%.

The cosmetic active ingredient according to the invention can also comprise ash, carbohydrates, and polyphenols.

The active ingredient according to the invention preferably comes in liquid form. It can be defined by at least one—preferably all—of the characteristics disclosed below.

### Dry Materials:

The level of dry materials of an active ingredient derived from Torulaspora delbrueckii according to the invention, measured by running it into the oven at 105° C. in the presence of sand until a constant weight is achieved, is between 10 and 150 g/l, preferably between 18 and 28 g/l.

### Measurement of pH:

The pH that is measured by the potentiometric method at ambient temperature leads to values of between 4.0 and 7.0, preferably between 5.3 and 6.3.

### Proteins

Determination of the Protein Content:

The metering of the protein content is done according to the LOWRY method (Lowry et al., Protein Measurement with the Folin Reagent, J. Biol. Chem., 193, 265-275, 1951). The folin reaction provides a blue coloring with certain amino acids. The coloring that is produced is compared to that of an established curve with a standard serum.

The total protein content is preferably between 6.5 and 107 g/l, and even more preferably between 12 and 20 g/l.

The protein content can also be expressed in terms of percentage relative to the dry material. It therefore preferably represents at least 42% by weight relative to the dry material.

### Characterization of the Protein Fraction:

The characterization of the protein fraction of the active ingredient according to the invention is carried out by steric-exclusion F.P.L.C. (Fast Protein Liquid Chromatography). The calibration of the column is done by the passage of markers of defined molecular weight (cytochrome C, aprotinin, vitamin B12, and cytidine).

The proteins of the active ingredient according to the invention consist of at least 60% of peptides of a molecular weight of less than 2,000 Da.

### Raw Ash Content:

The raw ash content is determined by weighing residues derived from incineration at 550° C. in an electric muffle furnace (VULCAN™).

The weight of the residue is calculated by subtracting the tare.

The raw ash content is expressed in terms of percentage relative to the dry material.

The raw ash content of an active ingredient according to the invention is preferably between 25 and 31%.

### Carbohydrates:

Determination of the Total Sugar Content:

The metering of the total sugar content can be carried out by the DUBOIS method (DUBOIS, M. et al., (1956), Analytical Chemistry, 28, No. 3, pp. 350-356). In the presence of concentrated sulfuric acid and phenol, reducing sugars provide an orangey-yellow compound. Starting from a standard range, it is possible to determine the total sugar level of a sample.

The total sugar content is preferably between 6.2 and 7.7% by weight relative to the dry material.

### Characterization of Carbohydrates:

The molecular weights of the carbohydrates that are present in the active ingredient according to the invention are determined using high-performance liquid-phase chromatography (HPLC).

The glucidic fraction of the active ingredient according to the invention consists of glucose, mannose and fructose, in the form of oligosaccharides with a degree of polymerization that is less than or equal to 20 (molecular weight that is less than or equal to 3,600 Da).

### Phenolic Compound Content:

The phenolic compounds form colored compounds, detectable at 715 nm, in the presence of potassium ferrocyanide. The coloring intensity is proportional to the quantity of phenolic compounds.

The readings are done starting from a standard range of gallic acid ranging from 40 to 120 mg/l. The results that are obtained for the standards make it possible to trace an optical density straight line based on the concentration, and the level of polyphenols of the samples is read directly on this straight line. The content of phenolic compounds of an active ingredient obtained from Torulaspora delbrueckii according to the invention is less than 0.4% as a percentage relative to the dry material.

### Identification of the Active Fraction

So as to determine the active fraction of the active ingredient according to the invention, a study was done. This study is done starting from the active ingredient of

### Example 1

It consists in fractionating the molecular species of the active ingredient according to the invention:
A fraction A that consists of ash, obtained by re-dissolving residues derived from incineration at 550° C. in an electronic muffle furnace, incorporated into the distilled and filtered water,
A fraction B essentially containing sugars, obtained by purifying the neutral carbohydrates of the active ingredient by successive adsorption of the cationic compounds and then anionic compounds on ionic resins,
A fraction C that essentially consists of peptides, obtained by purifying the peptides by adsorption/elution on a cation exchange resin.

The study consists in comparing the effect of these different fractions metered at 1% on the synthesis of loricrin, with the result obtained for the active ingredient according to the invention at 1%. The test is done on normal human keratinocytes, according to the operating procedure described in Item A.II of the part on the evaluation of the cosmetic effectiveness.

The results that are obtained are presented in the table below:

| | Ratio of Loricrin/Control (%) |
|---|---|
| Active Ingredient, 1% | +282 |
| Fraction A, 1% | +6 |
| Fraction B, 1% | 0 |
| Fraction C, 1% | +214 |

The fractions A and B are not effective. It is the fraction C that imparts to the active ingredient its activity.

The analysis of the fraction C by HPLC (high-performance liquid chromatography) shows that it consists of 86% peptides of which 71% have a molecular weight of less than 2,000 Da.

These are therefore essentially the peptides, in particular the peptides with a molecular weight of less than 2,000 Da, which impart to the active ingredient its effectiveness.

### Production Process

An active ingredient derived from Torulaspora delbrueckii according to the invention can be obtained by a process that makes it possible to obtain and to concentrate the active ingredient in terms of peptides, in particular in terms of peptides with a molecular weight of less than 2,000 Da. It comprises at least the following stages:
Aqueous solubilization of the Torulaspora delbrueckii fermenting agents, and
At least one enzymatic hydrolysis for the purpose of obtaining peptides with a molecular weight of less than 2,000 Da.

Preferably, it comprises at least the following stages:
Aqueous solubilization of the Torulaspora delbrueckii fermenting agents,
Separation of the soluble and insoluble phases for recovering the soluble phase,
At least one enzymatic hydrolysis for the purpose of obtaining peptides with a molecular weight of less than 2,000 Da, and
Deactivation of enzymatic activities.

According to a suitable embodiment, the process comprises at least the following stages:
Aqueous solubilization of the Torulaspora delbrueckii fermenting agents, preferably at a rate of 20 g/l,
Separation of the soluble and insoluble phases for recovering the soluble phase,
Enzymatic hydrolysis for obtaining peptides with a molecular weight of less than 2,000 Da,
Deactivation of enzymatic activities by heat treatment,
Filtering and concentration of the active fraction comprising peptides with a molecular weight of less than 2,000 Da.

Enzymatic hydrolysis is preferably carried out using a protease, under conditions that make it possible to obtain peptides with a molecular weight of less than 2,000 Da.

Additional stages of filtering and sterilizing filtering, color removal and/or deodorization can be considered.

The active ingredient that is obtained comes in the form of a clear liquid aqueous solution of a light yellow color.

### Cosmetic Compositions and Cosmetic Skin Care Process

This invention also covers the cosmetic compositions incorporating at least one active ingredient that is obtained from Torulaspora delbrueckii in different galenical forms, adapted to administration by topical cutaneous means.

These compositions can come in particular in the form of oil-in-water emulsions, water-in-oil emulsions, multiple emulsions (water/oil/water or oil/water/oil) that can optionally be microemulsions or nanoemulsions, or in the form of solutions, suspensions, hydrodispersion, aqueous gel, or powders. They can be more or less fluid and have the appearance of a cream, a lotion, a milk, a serum, an ointment, a gel, a paste, or a foam, or they can be in solid form.

These compositions contain between 0.01 and 20% by weight of active ingredient(s) derived from Torulaspora delbrueckii according to this invention, preferably between 0.1% and 3%.

These compositions comprise, in addition to the active ingredient, a medium that is physiologically acceptable and preferably cosmetically acceptable, i.e., that does not cause unacceptable feelings of discomfort for the user, such as redness, tingling, or prickling.

The compositions according to the invention can contain as adjuvant at least one compound that is selected from among:
The oils, which can be selected in particular from among the volatile or non-volatile, linear or cyclic silicone oils;
The waxes, such as ozokerite, polyethylene wax, beeswax, or carnauba wax;
Silicone elastomers;
Surfactants, preferably emulsifying surfactants, whether they are non-ionic, anionic, cationic, or amphoteric;
Co-surfactants, such as linear fatty alcohols;
Thickeners and/or gelling agents;
Moisturizers, such as polyols like glycerine;
Organic filters;
Inorganic filters;
Dyes, preservatives, feedstocks;
Tightening agents;
Sequestering agents;
Perfumes;
And mixtures thereof, without this list being limiting.

Examples of such adjuvants are cited in particular in the Dictionnaire CTFA (International Cosmetic Ingredient Dictionary and Handbook published by the Personal Care Product Council).

Of course, one skilled in the art will ensure that the optional complementary compounds, active or non-active, and their quantity are selected in such a way that the advantageous properties of the mixture are not—or essentially are not—altered by the addition being considered.

These compositions are intended in particular for the care, the treatment, and the protection of the human skin against the effects of various internal or external attacks, in particular for improving the barrier function and/or the surface condition of the skin.

For this purpose, the object of the invention is a cosmetic process for care of the human skin, intended to improve the barrier function and/or the surface condition of the skin, comprising the topical application on the skin of a composition that contains an active ingredient derived from Torulaspora delbrueckii, in particular a composition that contains between 0.01 and 20% by weight of active ingredient(s) derived from Torulaspora delbrueckii according to this invention.

### EXAMPLES

A nonlimiting example of a process for the production of an active ingredient derived from Torulaspora delbrueckii comprising peptides with a molecular weight of less than 2,000 Da is presented below, as well as examples of a composition incorporating such an active ingredient.

### Example 1

### Process for the Production of the Active Ingredient According to the Invention

An example of a process for the production of an active ingredient according to the invention comprises the implementation of the following stages:
Aqueous solubilization with 20 g/l of Torulaspora delbrueckii fermenting agents,
Separation of the soluble and insoluble phases for recovering the soluble phase,
Enzymatic hydrolysis for obtaining peptides with a molecular weight of less than 2,000 Da using a protease,
Deactivation of enzymatic activities by heat treatment,
Color removal by adding adjuvant,
Filtering so as to eliminate the molecules with high molecular weight,
Concentration of the active fraction comprising peptides with a molecular weight of less than 2,000 Da,
Filtering and sterilizing filtering on 0.22 μm.

The active ingredient that is obtained has the following characteristics:
Appearance: clear liquid
Color: light yellow
Dry material content: 25.1 g/l
pH: 5.8
Total protein content: 16.2 g/l, or 64.50% by weight relative to the dry material,
Total sugar content: 1.7 g/l, or 6.95% by weight relative to the dry material,
Ash content: 7.10 g/l, or 28.30% by weight relative to the dry material,
Polyphenol content of 0.25% by weight relative to the dry material.

### Example 2

### Use of an Active Ingredient According to the Invention in a Hydrating Gel

The formulation is as follows:

| | | |
|---|---|---|
| A. | Water | Enough to produce 100% |
| | Propylene glycol | 4% |
| | Carbopol Ultrez 10 (Lubrizol) | 0.3% |
| B. | Simulsol 1292DF (Seppic) | 4% |
| | DUB PTCC (Stéarinerie Dubois) | 6.3% |
| | Microcare C1024 (Thor) | 3.3% |
| | DUB OLEINE V2 (Stéarinerie Dubois) | 3.3% |
| | Montanov 14 (Seppic) | 3% |
| C. | Preservative | 0.7% |
| | Active ingredient according to the invention | 3% |
| D. | NaOH | Enough to produce pH 5.5 |

This emulsified gel has a pH of 5.5.

It can be obtained by the implementation of the following stages:
Mix A, disperse the gel thoroughly,
Mix B,
Heat A and B separately at 80° C. while stirring,
Emulsify B in A under an emulsifying agent,
Add C in order at 40° C. while stirring with a rotor-stator,
Adjust the pH with D at 30° C. slowly while ensuring good homogenization of the gel,
Leave it under an emulsifying agent until cooling is complete.

### Example 3

### Use of an Active Ingredient According to the Invention in a Night Cream

The formulation is as follows:

| | | |
|---|---|---|
| A. | Water | Enough to produce 100% |
| | Carbopol 2050 (Noveon) | 0.4% |
| B. | DUB MCT 5545 (Stéarinerie Dubois) | 4% |
| | DUB SEG (Stéarinerie Dubois) | 3% |
| | Cetyl alcohol (Stéarinerie Dubois) | 1% |
| | Sophiderm (Sophim) | 9% |
| | Beeswax (Rita) | 1% |
| | DUB DIOL (Stéarinerie Dubois) | 6% |
| | BIOPHILIC H (Lucas Meyer) | 2% |
| C. | Preservative | 0.7% |
| | Active ingredient according to the invention | 3% |
| D. | NaOH | Enough to produce pH 6 |

This creamy white emulsified gel has a pH of 6.

It can be obtained by the implementation of the following stages:
Mix A, disperse the gel thoroughly,
Mix B,
Heat A and B separately at 80° C. while stirring,
Emulsify B in A with an emulsifying rotor-stator at 1,000 rpm,
Add C in order at 40° C. while stirring with a rotor-stator,
Adjust the pH with D at 30° C., under a mechanical stirring mechanism at 1,500 rpm,
Leave it under an emulsifying agent until cooling is complete.

### Example 4

### Use of an Active Ingredient According to the Invention in a Gel

The formulation is as follows:

| | | |
|---|---|---|
| A. | Water | Enough to produce 100% |
| | Carbopol ETD 2020 (Noveon) | 0.2% |
| B. | Phoenate GC-7 (Phoenix Chemical) | 1.2% |
| | DUB MCT (Stéarinerie Dubois) | 0.8% |
| C. | Preservative | 0.7% |
| | Active ingredient according to the invention | 3% |
| D. | NaOH | Enough to produce pH 6.3 |

This gel can be obtained by the implementation of the following stages:
Mix A, disperse the gel thoroughly,
Mix B,
Emulsify B in A with an emulsifying rotor-stator at 1,800 rpm,
Add C in order while stirring with a rotor-stator,
Adjust the pH with D slowly while ensuring good homogenization of the gel.

### Evaluation of the Cosmetic Effectiveness of an Active Ingredient According to the Invention

These tests have as their object to illustrate the invention by showing the cosmetic effectiveness of the active ingredient according to the invention on the skin, in particular for repairing or improving the barrier function.

The active ingredient that is used for the tests is that of Example 1.

The composition that is used for the in-vivo tests is that of Example 4.

### A. In-Vitro Test

The effect of the active ingredient according to the invention on the differentiation and the adhesion of the keratinocytes and its mechanism of action via the nACHRs have been studied through the use of an antagonist and an agonist of the nAChRs evaluated on the synthesis of loricrin (on human keratinocytes in a single layer and on reconstructed epidermis) and E-cadherin (on reconstructed epidermis).

### I. Modeling

The procedure used to validate the agonist and the antagonist of the nAChRs is illustrated below for the synthesis of loricrin on reconstructed epidermis. It also applies to the synthesis of E-cadherin on reconstructed epidermis. The inserts comprising the epidermis under construction are placed in a culture medium containing:
1,000 μM of mecamylamine (reversible antagonist of the nACHRs) or
500 μM of nicotine (reversible agonist of the nACHRs).

On D18, the inserts are recovered, fixed, dehydrated, and incorporated into paraffin.

Cuts are made, and then an immunohistological marking of the loricrin is done.

Visualization is then done on a microscope coupled to an image analysis system. The intensity of the fluorescence (green color) is proportional to the synthesis of loricrin.

A quantitative analysis, carried out using Matlab® software, made it possible to obtain the following results:

| | Synthesis of Loricrin |
|---|---|
| Untreated Control | 100% |
| Mecamylamine, 1,000 μM | 53% |
| Nicotine, 500 μM | 113% |

It is noted that in the presence of mecylamine, the synthesis of loricrin is reduced relative to the untreated control whereas it is increased during treatment with nicotine.

The inhibiting effect of mecamylamine and the stimulating effect of nicotine were also displayed in the synthesis of human keratinocyte loricrin in a single layer and in the synthesis of E-cadherin on reconstructed epidermis.

The mecamylamine can therefore be used as an antagonist of the nAChRs, and nicotine can be used as an agonist of the nACHRs.

### II. Effect of the Active Ingredient on the Keratinocytic Differentiation

This study has as its objective to evaluate the effectiveness of an active ingredient according to the invention on keratinocytic differentiation by studying the synthesis of loricrin. It was accomplished by Western Blot on normal human keratinocytes that are isolated from foreskins, according to the operating procedure described below.

On D0, the human keratinocytes are inoculated and then incubated at 37° C.

On D1 and D4, the cells are treated with:
0.5% or 1% active ingredient according to the invention,
250 μM of nicotine, agonist of the nAChRs, positive control,
350 μM of mecamylamine, antagonist of the nAChRs, and
1% active ingredient according to the invention in the presence of 350 μM of mecamylamine.

The cells are then incubated at 37° C.

On D7, the cellular extracts are recovered, and the Western Blot metering is carried out.

The results that are obtained as a percentage relative to the negative control are presented below:

| | Ratio of Loricrin/ |
|---|---|
| | Control (%) |
| Mecamylamine, 350 μM | −57 |
| Nicotine, 250 μM | +51 |
| Active Ingredient According to the | +75 |
| Invention, 0.5% | |
| Active Ingredient According to the | +282 |
| Invention, 1% | |
| Active Ingredient According to the | −58 |
| Invention, 1%, + Mecamylamine, 350 μM | |

It is noted that the active ingredient according to the invention or the nicotine (positive control) increases the synthesis of loricrin by the human keratinocytes. Tested at 1%, it increases this synthesis by 282% under the conditions of the study.

Furthermore, it is noted that in the presence of mecamylamine, reversible inhibitor of the nAChRs, the increase in the synthesis of loricrin by the active ingredient is greatly reduced (58% reduction) proving the dependence on the pathway of the nACHRs.

The active ingredient according to the invention therefore makes it possible to stimulate the keratinocytic differentiation, and this effect for the most part is dependent upon the pathway of the nAChRs.

### III. Effect of the Active Ingredient on the Maturation of the Epidermis

This study has as its objective to evaluate the capacity of an active ingredient according to the invention to promote the maturation of the epidermis, or the construction of a cohesive and stratified epidermis.

This epidermal construction was evaluated using:
A terminal differentiation marker: loricrin, and
A marker involved in the formation of adhesive junctions: E-cadherin.

The operating procedure is described below.

On D0, the human keratinocytes are cultivated in a culture medium and then inoculated on an insert. The culture medium is changed every 2 days in the presence of the active ingredient according to the invention at 1% with or without mecamylamine at 1,000 μM.

On D13 and/or D18, the inserts are recovered, fixed, dehydrated, and incorporated into paraffin.

Next, cuts are made, and then an immunohistological marking of the loricrin or the E-cadherin is done.

The display is then produced on a microscope coupled to an image analysis system. The intensity of the fluorescence (green color) is proportional to the synthesis of loricrin or cadherin.

A quantitative analysis, carried out using Matlab® software, made it possible to obtain the following results:

| | Synthesis (%) | |
|---|---|---|
| | Loricrin | E-Cadherin |
| Control | 100 | 100 |
| Active Ingredient | 122 | 143 |
| According to the Invention, 1% | | |
| Active Ingredient | 105 | 83 |
| According to the Invention, | | |
| 1%, + Mecamylamine, | | |
| 1,000 μM | | |

It is noted that the active ingredient according to the invention accelerates the construction of an epidermis and promotes the cohesion and maturation thereof by stimulating the synthesis:
of the loricrin, marker of the keratinocytic terminal differentiation, and
of the E-cadherin, protein involved in the formation of adhesive junctions.

Tested at 1%, the active ingredient according to the invention under the conditions of the study makes it possible to increase the synthesis of loricrin by 22% and the synthesis of E-cadherin by 43%.

In addition, in the presence of mecamylamine, the reversible inhibitor of the nAChRs, the effectiveness of the active ingredient according to the invention is reduced, which clearly shows that this effect for the most part depends on the nAChRs.

### A. In-Vivo Tests

### I. Study of the Capacity to Preserve the Barrier Function

The objective of the study is to quantify in vivo on volunteers the effect of the active ingredient according to the invention that is formulated at 3% in emulsified gel on the negligible water loss (NWL) of the skin in comparison to the placebo after an artificial disruption of the barrier function using a detergent.

The cutaneous barrier plays a regulating role in the balance of water of the skin. When the latter is damaged, misalignments in the regulation of the exchanges of water appear. The water then migrates more easily toward the outside environment, which increases the NWL. By contrast, if the state of the cutaneous barrier improves, the water loss values decrease because the regulation of exchanges of water is ensured in a correct way.

This study was done on the arms of 19 healthy female volunteers having normal skin.

The measurements were made using a Tewameter®, a device equipped with a probe that measures the vapor gradient that is installed between the cutaneous surface and the ambient air. This measurement makes it possible to assess the exchanges of water between the cutaneous surface and the surrounding environment.

The operating procedure of the study is described below.

Between D14 and D0, no cream was applied to the arms.

On D0, measuring zones at the tops of the arms were determined on each volunteer. The measuring zones are as follows:
Untreated zone
Zone treated with a placebo
Zone treated with a 3% active ingredient according to the invention.

NWL measurements are then made on each zone with a Tewameter®.

Between D0 and D6, the zones being studied are washed with an irritant soap, sodium lauryl sulfate (SLS), and twice daily, the volunteers also apply the product containing the active ingredient according to the invention or the placebo.

On D7, measurements of the NWL are made on each zone with the Tewameter®.

Between D7 and D13, the zones being studied are washed with an irritant soap, sodium lauryl sulfate (SLS), and twice daily, the volunteers also apply the product containing the active ingredient according to the invention or the placebo.

On D14, last measurements of the NWL are made on each zone with a Tewameter®.

The average of the results that are obtained with the active ingredient according to the invention is presented in the table below as a percentage of variation relative to the placebo:

| | Variation/Placebo |
|---|---|
| D7 | −5.9% |
| D14 | −11.0% |

Under the conditions of this study, it is noted that after 7 days of twice-daily applications, the active ingredient according to the invention that is formulated at 3% in emulsified gel reduces the NWL after being attacked repeatedly with 5.9% SLS by comparison to the placebo.

This result continues after 14 days of study (11% reduction of the NWL).

These results clearly show that an active ingredient that is obtained from Torulaspora delbrueckii according to the invention makes it possible to limit the alteration of the barrier function and thus to preserve the integrity of the stratum corneum.

### II. Study of the Capacity to Repair the Previously-Altered Barrier Function

a) Measurement of the NWL

The objective of the study is to quantify in vivo on volunteers the effect of the active ingredient according to the invention that is formulated at 3% in emulsified gel on the negligible water loss (NWL) of the skin in comparison to the placebo on a skin of which the barrier function was previously artificially disrupted by a detergent, sodium lauryl sulfate (SLS).

The study was done on the calves of 19 healthy female volunteers who have normal skin.

The measurements were made using a Tewameter®.

The operating procedure of the study is described below.

Between D14 and D7, no cream was applied on the arms.

Between D7 and D1, the volunteers apply an irritant soap (SLS) on the zones being studied.

On D0, measuring zones on the calves were determined on each volunteer. The measuring zones are as follows:
Untreated zone
Zone treated with placebo
Zone treated with a 3% active ingredient according to the invention.

Measurements of the NWL are then made on each zone with a Tewameter®.

Between D0 and D6, twice daily, the volunteers also apply the product that contains the active ingredient according to the invention or the placebo on the zones being studied.

On D7, measurements of the NWL are made on each zone with a Tewameter®.

Between D7 and D13, twice daily, the volunteers also apply the product that contains the active ingredient according to the invention or the placebo on the zones being studied.

On D14, last measurements of the NWL are made on each zone with a Tewameter®.

The average of the results that are obtained with the active ingredient according to the invention is presented in the table below as a percentage of variation relative to the placebo:

| | Variation/Placebo |
|---|---|
| D7 | −5.4% |
| D14 | −11.0% |

Under the conditions of this study, it is noted that after 7 days of twice-daily applications, the active ingredient according to the invention that is formulated at 3% in emulsified gel reduces the NWL of a skin having a barrier function that is altered with SLS by 5.4% in comparison to the placebo.

This effect continues after 14 days of study (11% reduction of the NWL).

These results clearly show that the application on the skin of an active ingredient that is obtained from Torulaspora delbrueckii according to the invention leads to a reduction in the losses in water of the skin, thus making it possible to restore the barrier effect of previously altered skin.

b) Evaluation of the Surface Condition of the Skin Observed on the Cellular Level

The objective of the study is to quantify in vivo on volunteers the effect of the active ingredient according to the invention that is formulated at 3% in emulsified gel on the surface condition of the skin observed on the cellular level by confocal microscopy, in comparison to the placebo on the skin whose barrier function was previously artificially disrupted by a detergent, sodium lauryl sulfate (SLS).

The study was done on the calves of 19 healthy female volunteers who have normal skin.

The acquisitions (corresponding to horizontal cuts of the skin) have been made using a confocal microscope on the volunteers on the stratum corneum after local application of fluorescein. Fluorescein acts as a contrast agent. It diffuses into the tissues and the interstitial spaces freely and does not specifically bond. It thus intermixes in the cells of the stratum corneum and accumulates in the thicker zones such as the squamae. After cleaning excess fluorescein, the acquisition is made using a 445 nm laser in fluorescence mode. The images that are obtained are next treated and then analyzed blind by a trained jury, according to the parameters indicated in the table below:

| | Presence | | | Arrangement | |
|---|---|---|---|---|---|
| | of | Shape oft he Cells | | of the Cells | |
| | Squamae | Hexagonal | Rounded | Honeycomb | Flake |
| Stage 1 | ++++ | − | ++ | − | ++ |
| Stage 2 | +++ | +/− | + | +/− | + |
| Stage 3 | ++ | + | +/− | + | +/− |
| Stage 4 | + | ++ | − | ++ | − |

Altération de la fonction barrière = Alteration of the Barrier Function

The operating procedure of the study is described below.

Between D14 and D7, no cream was applied on the calves.

Between D7 and D1, the volunteers apply an irritant soap (SLS) on the zones being studied.

On D0, measuring zones on the calves are determined on each volunteer. The measuring zones are as follows:
Untreated zone
Zone treated with a placebo
Zone treated with a 3% active ingredient according to the invention.

Next, NWL measurements are made on each zone with a Tewameter®.

Between D0 and D6, twice daily, the volunteers also apply the product that contains the active ingredient according to the invention or the placebo on the zones being studied.

On D7, measurements of the NWL are made on each zone with a Tewameter®.

Between D7 and D13, twice daily, the volunteers also apply the product that contains the active ingredient according to the invention or the placebo on the zones being studied.

On D14, last measurements of the NWL are made on each zone with a Tewameter®.

The average of the results obtained with the active ingredient according to the invention is presented in the table below as a percentage of variation relative to the placebo:

| | Variation/Placebo |
|---|---|
| D7 | +19.9% |
| D14 | +25.7% |

Under the conditions of this study, it is noted that after 7 days of twice-daily applications, the active ingredient according to the invention that is formulated at 3% in emulsified gel improves the surface appearance of the skin by 19.9% in comparison with the placebo.

This effect continues after 14 days of study (improvement of 25.7% of the NWL).

These results clearly show that the application on the skin of an active ingredient obtained from Torulaspora delbrueckii according to the invention makes it possible to restore the cellular organization of the surface layer of the skin.

1. A cosmetic active ingredient, comprising peptides obtained from Torulaspora delbrueckii.

2. The cosmetic active ingredient according to claim 1, wherein said peptides have a molecular weight of less than 2,000 Da.

3. The cosmetic active ingredient according to claim 1, wherein said ingredient is a Torulaspora delbrueckii hydrolyzate.

4. The cosmetic active ingredient according to claim 3, wherein said hydrolyzate is obtained by enzymatic hydrolysis of Torulaspora delbrueckii.

5. The cosmetic active ingredient according to claim 1, wherein said ingredient comprises a peptide content that is greater than or equal to 42% relative to the total content of dry material.

6. The cosmetic active ingredient according to claim 1, wherein said ingredient has a level of dry material of between 10 and 150 g/l.

7. The cosmetic active ingredient according to claim 1, wherein said ingredient has at least one of the following characteristics:
a level of dry material of between 18 and 28 g/l,
a raw ash content of between 25 and 31% by weight relative to the dry material,
a total sugar content of between 6.2 and 7.7% by weight relative to the dry material.

8. A method for improving the barrier function and/or the surface condition of the skin, comprising topically applying to the skin an active ingredient according to claim 1 and/or a composition comprising said active ingredient.

9. A method of stimulating and/or increasing the expression of nicotinic acetylcholine receptors of the cells of the skin, comprising topically applying to the skin an active ingredient according to claim 1 and/or a composition comprising said active ingredient.

10. A method of stimulating the interkeratinocytic adhesion in the epidermis, comprising topically applying to the skin an active ingredient according to claim 1 and/or a composition comprising said active ingredient.

11. Stimulating the differentiation of keratinocytes in the epidermis, comprising topically applying to the skin an active ingredient according to claim 1 and/or a composition comprising said active ingredient.

12. A cosmetic composition for topical application, comprising between 0.01 and 20% by weight of an active ingredient according to claim 1.

13. A process for caring for human skin by improving and/or restoring the barrier function of the skin, comprising the topical application on the skin of a composition according to claim 12.
